(19) United States
(12) Patent Application Publication
(10) Pub. No.: US 2007/0253907 A1
(43) Pub. Date: Nov. 1, 2007

(54) Non Human Transgenic Animal as Model of Neurodegenerative Diseases and for the Early Diagnosis Thereof

(76) Inventors: Antonino Cattaneo, Roma (IT); Simona Capsoni, Roma (IT)

Correspondence Address: JUNEAU PARTNERS, P.O. BOX 2516, ALEXANDRIA, VA, 22301, US

(21) Appl. No.: 11/579,108

(22) PCT Filed: Apr. 29, 2005
(86) PCT No.: PCT/IT05/00249

§ 371 Date: Feb. 25, 2007

(30) Foreign Application Priority Data
Apr. 30, 2004 (IT) RM2004A000212

(51) Int. Cl. A01K 67/027; A01K 67/02; C12N 15/00; G01N 33/50

(52) U.S. Cl. 424/9.2; 436/513; 800/12; 800/18; 800/22; 800/9

(57) ABSTRACT

A non human transgenic animal able to express ubiquitarily an anti-NGF neutralizing antibody wherein said anti-body is composed by an endogenous VH chain and by an exogenous VK chain; uses as an animal model to identify compounds with therapeutic activity, in particular for neurodegenerative pathologies. Method for the early prognosis and/or diagnosis of neurodegenerative diseases comprising the drawing of a peripheral biological fluid from a patient and the detection in said fluid of antibodies anti-NGF, or anti-TrkA or antibodies against proteins linked to NGF activity.

The present invention relates to a non human transgenic animal as a model for neurodegenerative diseases and for their early diagnosis

Introduction

The study of NGF (Nerve Growth Factor) action can be conducted by means of animal models in which the action of NGF is blocked by neutralizing anti-NGF antibodies (Angeletti and Levi-Montalcini, 1971; Gorin and Johnson, 1979, 1980; Molnar et al., 1998) or by knockout of the gene that synthesizes NGF (Crowley et al., 1994; Chen et al., 1997).

The approach of producing a transgenic mouse that expresses recombinant antibodies neutralizing NGF (Ruberti et al., 2000, PCT application WO01/10203) has highlighted two results. In the first place, the inactivation of NGF by means of neutralizing recombinant antibodies has allowed to provide a model for studying the effects of NGF neutralization on adult organisms: the gene knockout approach did not allow to do so, because ngf^{-/-} mice die shortly after birth, without any chance for any neurodegenerative diseases connected to aging to develop (Crowley et al., 1994). The second result consists of actually producing an animal model for one of the most common neurodegenerative diseases among the elderly, i.e. Alzheimer's disease (Capsoni et al., 2000a; Capsoni et al., 2000b; Capsoni et al., 2002a, b, c; Pesavento et al., 2002). The fact that Alzheimer's disease was reproduced in mice can be linked to 2 factors: (i) the neutralization of NGF (ii) the introduction of an antibody that neutralizes an endogenous protein in mice's organism.

Different experimental evidences suggest that NGF can play an important role in Alzheimer's disease. This pathology is characterized by progressive dementia which affects the elderly with an incidence exceeding 30% in patients over 80 years of age. The incidence of the disease, linked to the progressive increase in life expectancy, is destined to double over the next 30-50 years. Since there is no therapy, the disease has extremely high social costs.

The etiology of Alzheimer's disease is unknown and its immediate causes may be many and reside not only in the encephalon but also in non nervous tissues of the body's peripheral regions, since cells of the immune, hematopoietic and circulatory systems appear to be altered in patients affected by Alzheimer's disease (Gasparini et al., 1998). In particular, there is a hypothesis that one of the factors causing neurodegeneration could be auto-antibodies which trigger an auto-immune or auto-toxic reaction (McGeer and McGeer, 2001).

Since cholinergic neurons of the basal forebrain express NGF receptors, it has been hypothesized that deficits in the retrograde transport and alterations in the signal transduction of the NGF/receptor system may be one of the possible causes of Alzheimer's disease.

To date, there is no early diagnosis or therapy for the disease due to the lack, up to a short time ago, of experimental cellular or animal models that reproduce the disease in a complete fashion. Transgenic mice that produce the mutated forms of the amyloid precursor protein, APP, the hyperphosphorylated form of tau or the mutated forms of presenilin 1 or 2 (Gotz, 2001; Janus et al., 2001) do not reproduce all characteristics of Alzheimer's disease. The attempt to obtain a complete model by crossing transgenic mice that express different mutated proteins linked to Alzheimer's disease, while allowing to obtain mice with larger neurodegenerative lesions than in parental mice, failed because they express the mutated proteins independently from an overall pathological process, and in any case they do not exhibit cholinergic deficits nor significant cell death (Borchelt et al., 1997; Oddo et al., 2003). The most complete model of the disease was obtained through the expression of NFG neutralizing recombinant antibodies (alfaD11, Cattaneo et al., 1988). These mice are characterized by the presence of behavioral deficits (Capsoni et al., 2000b) and synaptic plasticity deficits (Pesavento et al., 2002), events linked to loss of cholinergic neurons, neuron loss in the cortex, tau hyperphosphorylation with formation of intracellular tangles, deposit of β-amyloid plaques (Capsoni et al., 2000a; Capsoni et al., 2000b; Capsoni et al., 2002a; b; c).

These mice's Alzheimer's phenotype demonstrates that an Alzheimer's-type neurodegeneration is induced by blocking NGF activity. This could have relevance for the situation in humans.

AD11 anti-NFG mice, which express the functional form of the αD11 monoclonal antibody, were produced by crossing mice that express the heavy chain of the transgenic antibody (VH-AD11 mice) with mice that express the light chain of the antibody (VK-AD11 mice). “Exogenous chains” means the VH and VK transgenic antibody chains of the αD11 recombinant antibody, whereas “endogenous chains” means the antibody chains of the antibodies produced by the mouse's lymphocytes. In spite of the advantages obtained with this technique, having to continuously re-cross the mice of the two lines VH-AD11 and VK-AD11 requires having to maintain 3 lines of animals, instead of a single one. Another disadvantage is the poor reproductive ability of anti-NGF AD11 mice. Indeed, crossing different transgenic mice with each other is a useful experimental procedure, because it enables to obtain information on the combined activities of the transgenes of the parental lines, thereby generating new experimental models. In the case of anti-NGF AD11 mice, this possibility of crossing with other transgenic mice is made difficult, if not impossible, since anti-NFG AD11 mice have poor reproductive ability.

### DESCRIPTION OF THE INVENTION

The authors of the invention have surprisingly found that VK-AD11 mice, which express a single transgenic chain VK, in the absence of the corresponding transgenic chain VH, exhibit a complex neurodegenerative picture, similar to that of anti-NGF AD11 mice. This occurs because the exogenous light chain of the recombinant antibody is assembled with an endogenous heavy chain of mouse IgG, to yield a functional NGF neutralizing antibody. It is noteworthy that VH-AD11 mice have no phenotype linked to a neurodegenerative picture. Finally, the authors have shown that VK-AD11 mice reproduce effectively.

According to the invention, an improvement is obtained in the procedure for obtaining a transgenic mouse, which is a complete and unique model for Alzheimer's disease, and for assessing the implications of an alteration at the level of the immune system in the emergence of the disease. Indeed, the heavy chain of an endogenous antibody cannot assemble with the light chain of an antibody, except in lymphocytes (Abbas et al., 2000). Therefore, the cerebral alterations observed in the mouse described in this invention can only be due to antibodies produced first in the blood and hence can only be secondary to alterations of the hematoencephalic barrier which allows the passage of the transgenic antibodies and/or of eventual cells of the immune system from the periphery to the central nervous system. Therefore, VK-AD11 mice allow to analyze the peripheral alterations (and in particular antibodies produced by peripheral lymphocytes), able to determine the onset in the central nervous system of a neurodegeneration similar to Alzheimer's disease. Thus this result suggests a method for the early diagnosis of the disease, based on the determination in biological samples of Alzheimer's patients of antibodies directed against NGF or proteins required for its mechanism of action. These characteristics are absent in other animal models for Alzheimer's disease and consequently the mice described in the present invention represent a unique model to study the importance of these components in the etiology of the disease and to develop early diagnostic methods.

### DETAILED DESCRIPTION OF THE INVENTION

Therefore, the object of the invention is a non human transgenic animal able to express ubiquitarily an anti-NGF neutralizing antibody in which the antibody is composed by an endogenous VH chain and by an exogenous VK chain. Preferably, the exogenous VK chain is that of the anti-NGF AD11 antibody, having essentially the amino acid sequence of SEQ ID No. 1, as follows:

aD11 VK human Ck
DIQMTQSPASLSASLGETVTIECRASEDIYNALAWYQQKPGKSPQLLIYN
TDTLHTGVPSRFSGSSGTQYSLKINSLQSEDVASYFCQHYFHYPRTFGGG
TKLELKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

In a preferred embodiment, the non human transgenic animal belongs to the murine genus, preferably to the Mus musculus species.

The object of the invention is the use of the non human transgenic animal as an animal model for identifying compounds with therapeutic activity for pathologies, in particular neurodegenerative pathologies.

Further object of the invention is the use of the non human transgenic animal for crossing with a second non human transgenic animal for at least one other function involved in pathologies, also neurodegenerative, and obtaining a line of non human transgenic animals with at least two transgenes, in which said transgenes codify for functions involved in pathologies, also neurodegenerative. Preferably, the second non human transgenic animal is homozygote “knockout” for the gene of the NGF receptor, p75NTR or parts thereof.

The scope of the invention further includes a method for the early prognosis and/or diagnosis of neurodegenerative diseases comprising the drawing of a peripheral biological fluid from a patient and the detection in said fluid of antibodies anti-NGF, or anti-TrkA or against proteins linked to NGF activity. Preferably, the peripheral biological fluid is blood, serum or urine. Preferably, the neurodegenerative disease is Alzheimer's Disease.

The present invention describes a non human transgenic animal that expresses an antibody neutralizing the Nerve Growth Factor (NGF). The antibody used is constituted by the endogenous heavy chain of IgG and by the light chain of the αD11 recombinant antibody. The αD11 antibody specifically binds NGF at the epitope responsible for its binding with its high affinity receptor, TrkA. Consequently, the anti-NGF antibody blocks the binding of NGF to its receptor and neutralizes its activity.

Transgenic mice that express this anti-NGF antibody (VK-AD11 mice) develop antibody levels ranging between 50 and 500 ng/ml in adult age, and develop a complex pathological picture whose characteristics are summarized as:
1) dilation of the cerebral ventricles;
2) atrophy of the cerebral cortex associated to atrophy of the hippocampus;
3) loss of neurons and apoptosis;
4) deposition of β-amyloid plaques in the hippocampus and cerebral cortex;
5) neurofibrillary tangles;
6) tau hyperphosphorylation at the cerebral level;
7) aggregation of the tau protein at the cerebral level;
8) cognitive deficit characterized by “working memory” deficits and deficit in terms of spatial orientation;
9) cholinergic deficit in the basal forebrain and Meynert's nucleus;
10) alternations of sympathetic innervations of the cerebral arteries;
11) alterations of the permeability of the hematoencephalic barrier;
12) decrease in the volume and number of neurons in the upper cervical ganglia.

Many of the characteristics described in this transgenic model are wholly similar to those present in Alzheimer's disease. The VK AD11 model therefore is suitable for use as an instrument for etiologic research and for the experimentation of new potential therapeutic agents and diagnostic means. A further aspect of this invention relates to the use of VK-AD11 mice to produce new mice deriving from the crossing of these mice with other transgenic mice.

### DESCRIPTION OF THE FIGURES

FIG. 1. Transcriptional unit used for the production of the VK-AD11 transgenic mouse. CK constant human region, VL variable regions of the light chain of the αD11 monoclonal antibody; pCMV promoter of the human Cytomegalovirus.

FIG. 2. PCR analysis of VK transgenic mice.

FIG. 3. (A) Levels of recombinant antibody in 60 day old adult mice, measured in serum by incubation with an antibody anti human light chain and anti heavy chain of mouse IgG. The antibody anti mouse light chain does not show the presence of cross-reactivity. (B) Levels of transgenic antibody in the serum and in the cerebral tissue, quantified by comparison with a standard curve. The dotted line indicates the limit of detection of the assay (0.1 ng/ml and 0.1 ng/mg, respectively).

FIG. 4. The images show the representation of: (A) an NGF neutralizing antibody constituted by an exogenous VH chain and a transgenic VK chain; (B) an antibody constituted by a heavy endogenous chain and a transgenic VK chain.

FIG. 5: Expression of the VK light chain of the transgenic antibody in the cerebral cortex of the VK-AD11 mouse. (B) Absence of the expression of the VK light chain of the recombinant antibody in WT mice.

FIG. 6. Body weight of the VK-AD11 mouse and of the control mouse at two months of age.

FIG. 7. Reduced area of the median sagittal section of the upper cervical ganglion in the VK-AD11 mouse (A) with respect to the one observed in the control mouse (B).

FIG. 8. (A) Sympathetic innervations of the basilar artery of VK-AD11 mouse is decreased with respect to what is observed (B) in the control mouse.

FIG. 9. The chart shows the increase in Evans Blue concentration in the cerebral tissue due to the breaking of the hematoencephalic barrier.

FIG. 10. Atrophy of the cerebral cortex and of the hippocampus in VK-AD11 mice. The measurements were obtained from coronal sections of the mouse encephalon at the level of the parietal cortex and of the antero-dorsal part of the hippocampus.

FIG. 11. Progressive decrease in the total number of cholinergic neurons in the basal forebrain of VK-AD11 mice with respect to control mice of the same age.

FIG. 12. Decrease in the number of cholinergic neurons in the basal forebrain is particularly marked in the medial septum (MS).

FIG. 13. (A) Tau hyperphosphorylation in the hippocampus of 6 month old VK-AD11 mice. (B) Tau hyperphosphorylation in the cerebral cortex of 6 month old VK-AD11 mice. (C) Presence of tangle-like formations in 15 month old VK-AD11 mice. (D) Absence of staining with the mAb AT8 antibody in control mice of the same age.

FIG. 14. Presence of protofibrils of phosphorylated tau, marked with the antibody AT8 and comprised in the tangles, in the VK-AD11 mouse.

FIG. 15. (A) Enlargement of β-amyloid plaques marked with the monoclonal antibody 4G8 in 15 month old VK-AD11 mice. (B) Absence of plaques in control mice of the same age.

FIG. 16. (A) Spatial orientation test conducted in VK-AD11 mice and in control mice at 8 months of age.

FIG. 17. Object discrimination test in VK-AD11 mice and in the control mice conducted at 6 months of age.

FIG. 18. The treatment with NGF administered intranasally improves (A) the cholinergic deficit, (B) decreases the number of cells containing β-amyloid and (C) the number of cells that express phosphorylated tau.

FIG. 19. The table shows the better reproductive ability of VK-AD11 mice with respect to AD11 mice.

FIG. 20. Validity of the use of VK-AD11 mice with respect to AD11 mice to obtain transgenic mice.

FIG. 21. Outline of the method for diagnosing Alzheimer's disease, based on the presence of anti-NGF antibodies or antibodies directed against NGF-correlated proteins.

FIG. 22. Presence of anti-NGF and anti-TrkA antibodies in the serum of patients affected by Alzheimer's disease.

### EXAMPLE 1

### Production and Characterization of the VK-AD11 Transgenic Mouse

Production of AD1 VK Mice

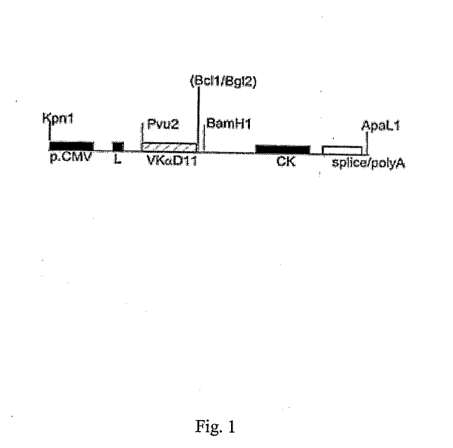

The VK-AD11 mice were obtained from the injection into the pronucleus of fertile eggs of C57BL/6×SJLF2 hybrid mice of the plasmide pcDNA-neo/VKαD11HuCK containing the transcriptional unit of the gene of the light chain of the αD11 transgenic antibody (FIG. 1) conducted according to standard methods (Alle et al., 1987). Crossing heterozygote mice allowed to obtain two lines of homozygous mice (line A and line B) that express the VK-AD11 chain in different quantities. The mice are fertile and the lines were brought to homozygosity.

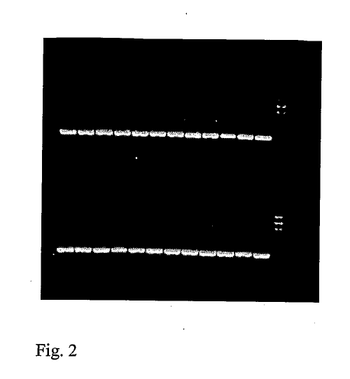

Molecular analysis of the mice was performed by PCR on genomic DNA extracted from tail biopsies (FIG. 2A).

Characterization of the Transgenic Antibody

The presence of a chimeric antibody obtained from the assembly of an endogenous heavy chain of IgG with the light chain of the αD11 recombinant antibody was verified by ELISA of the sera and of the extracts of VK-AD11 transgenic mice.

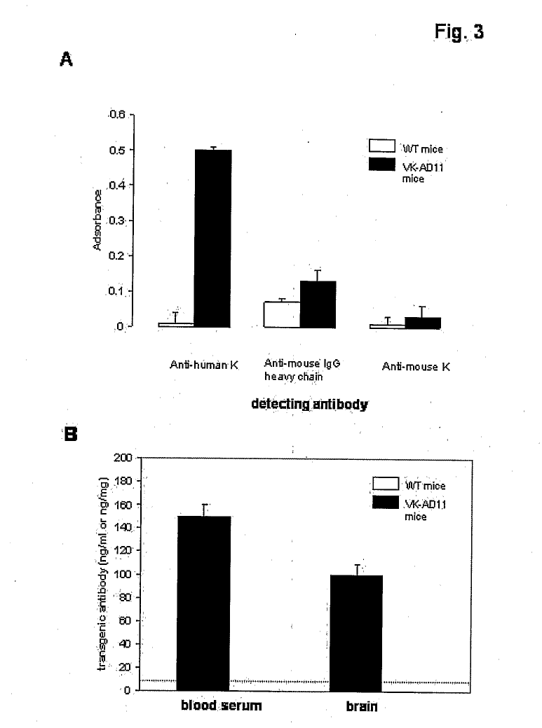

The plate for ELISA was incubated with NGF (5 μl/ml) and the transgenic antibody was made to bind to NGF. The recognition of the antibody is possible both with a specific biotinylated for the murine heavy chain of IgG and with a specific antibody for the human light chain of IgG. Both antibodies recognize the transgenic antibody linked to NGF (FIG. 3A).

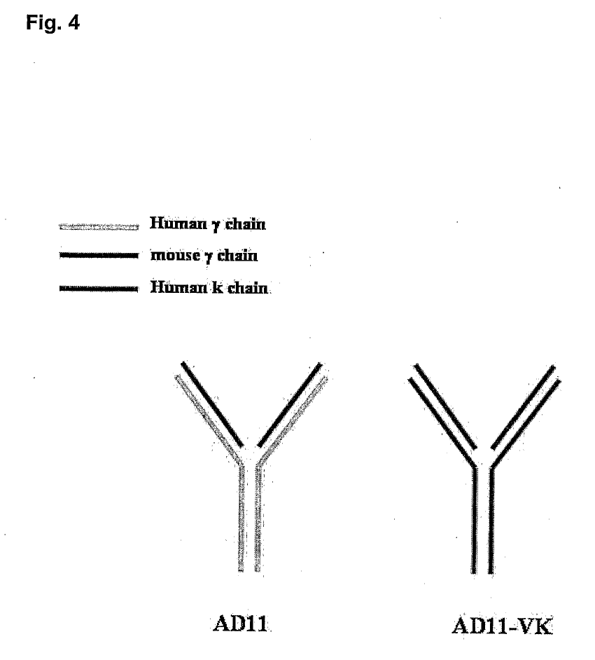

The level of anti-NGF chimeric antibody measured in the serum and in the cerebral tissue of the A and B mice lines exceed 100 ng/ml and 100 ng/mg. In the adult mouse, antibody levels are greater by three orders of magnitude than the antibody level detected in mice aged between 1 and 30 days (0.1 ng/ml in serum and 0.1 ng/mg in cerebral tissue) (FIG. 3B). Therefore, the conclusion is that NGF is recognized both by the antibody composed by the two transgenic chains VH and VK (FIG. 4A), and by the hybrid antibody constituted by the endogenous heavy chain and by the transgenic VK chain (FIG. 4B).

Phenotypic Characterization of the VK-AD11 Mouse

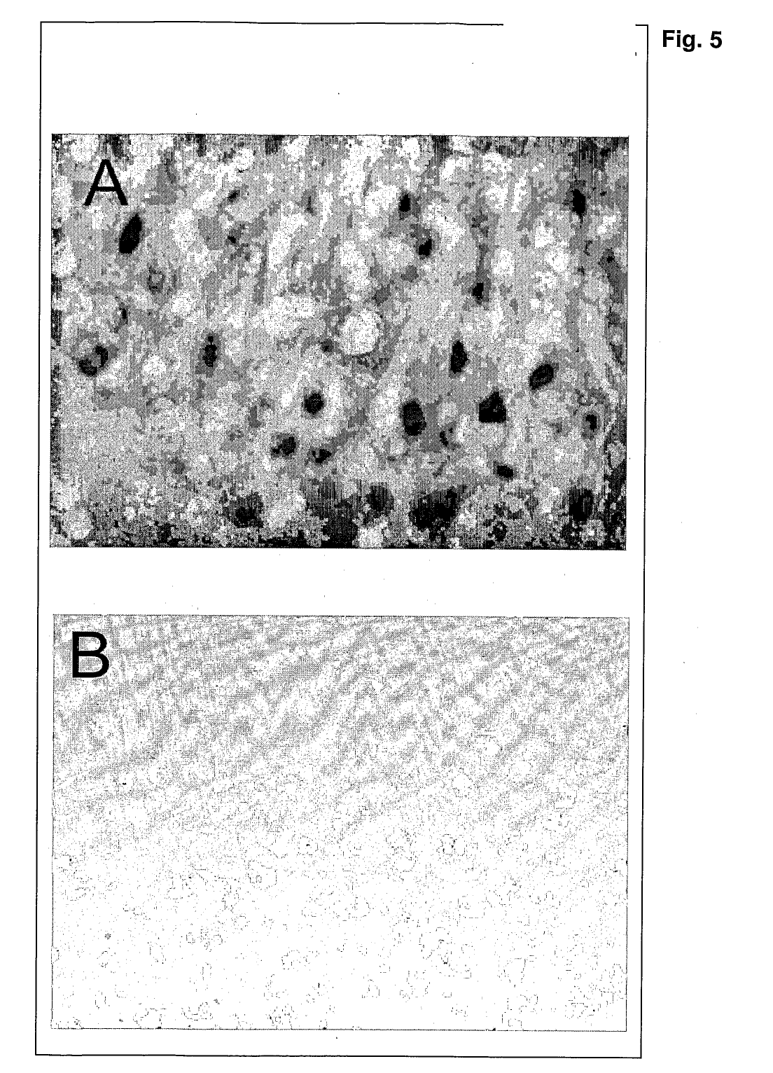

The tissues of the VK-AD11 mice were fixed by intracardiac perfusion of 4% paraformaldehyde in PBS, cryoprotected in 30% saccharose, and then sectioned. The sections were preincubated in 10% bovine fetal serum and then processed with immunohistochemical technique to detect the presence of the light chain of the recombinant antibody in the cerebral cortex of the VK-AD11 mice (FIG. 5).

### EXAMPLE 2

### Phenotypic Knock-Out of the NGF in the VK-AD11 Transgenic Mouse

Phenotypic characterization of the VK-AD11 mouse was conducted by macroscopic analysis and immunohistochemistry techniques. The experiments were conducted in groups of ten (n=10) with animals having antibody levels of 50-400 ng/ml. Normal, non transgenic mice of the same strain were used as controls.

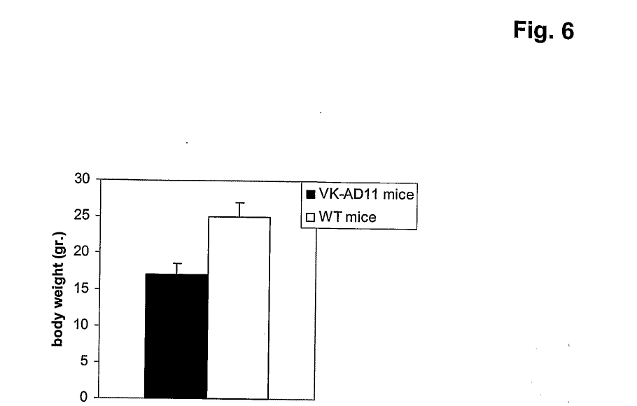

At macroscopic level, VK-AD11 mice do not exhibit relevant abnormalities during the first 4-6 weeks of life. However, a slowdown in growth is observed which is translated into a 20% decrease in body weight with respect to the control mouse (FIG. 6).

At the histological level, the following differences were observed with respect to normal mice: (1) reduced area of the upper cervical ganglion; (2) increased permeability of the hematoencephalic barrier; (3) reduced sympathetic innervation of the cerebral arteries; (4) reduced cholin-acetyltransferase synthesis; (5) atrophy of the cerebral cortex and of the hippocampus (6) hyperphosphorylation of the tau protein and presence of intracellular tangles of tau protein; (7) presence of β-amyloid plaques; (8) behavioral deficits.

(1) Reduced Area of the Upper Cervical Ganglion.

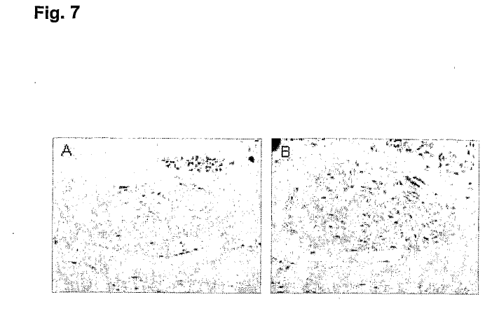

At the level of the peripheral nervous system, the upper cervical ganglia are smaller than in the control, with a 25% reduction in the surface of the mean section. The number of cells is also reduced by 50% (FIG. 7).

(2) Reduced Sympathetic Innervation of the Cerebral Arteries.

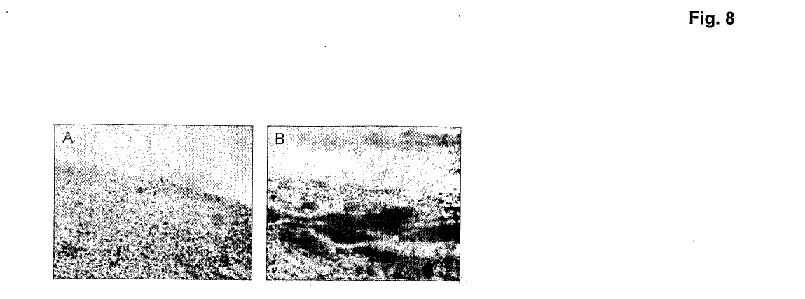

The sympathetic innervation of the cerebral arteries is strongly reduced in VK-AD11 mice with respect to control mice, as demonstrated by the reduced expression of the tyrosine hydroxylase marker protein (FIG. 8), measured by means of the anti-tyrosine hydroxylase antibody (Chemicon).

(3) Increased Permeability of the Hematoencephalic Barrier

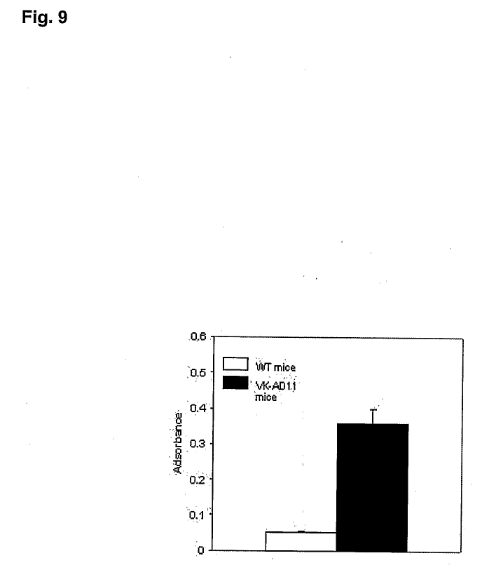

An increase in the permeability of the hematoencephalic barrier is observed after injection of the Evans Blue coloring substance, a marker whose presence is measured by spectrophotometry after intravenous injection into the mice. An increase in the quantity of colorant indicates an increase in the permeability of the hematoencephalic barrier to proteins (among them the antibodies) that normally do not pass through it (FIG. 9).

(4) Atrophy of the Cortex and of the Hippocampus in VK-AD11 Mice

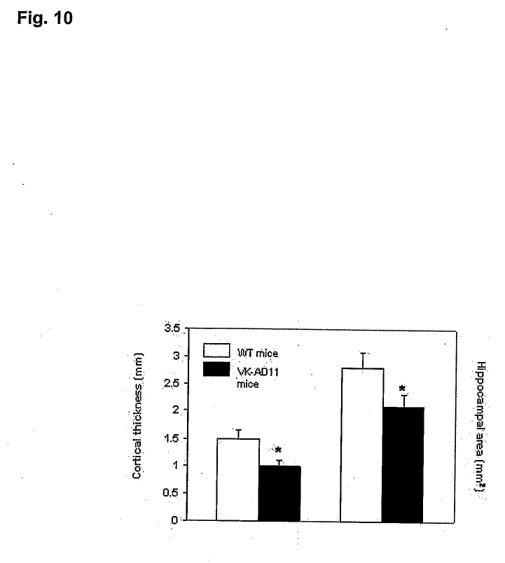

The analysis of the morphological aspect of the brain of VK-AD11 mice was conducted at 15 months of age and it revealed the presence of a marked atrophy of the cerebral cortex and of the hippocampus (FIG. 10).

(5) Reduction in Cholin-Acetyltransferase Synthesis in the Basal Forebrain.

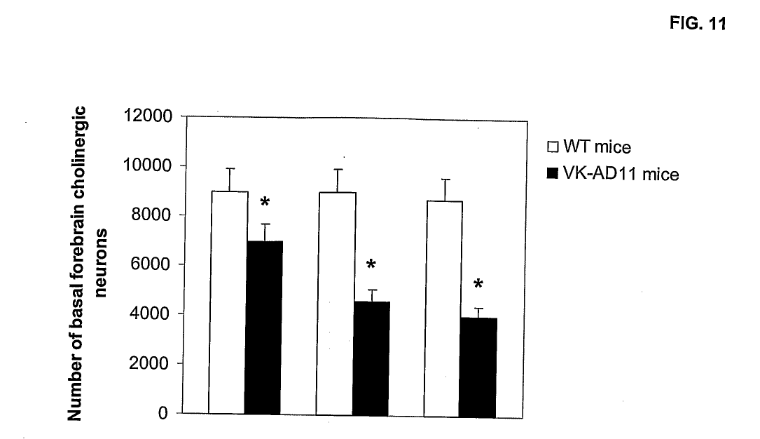

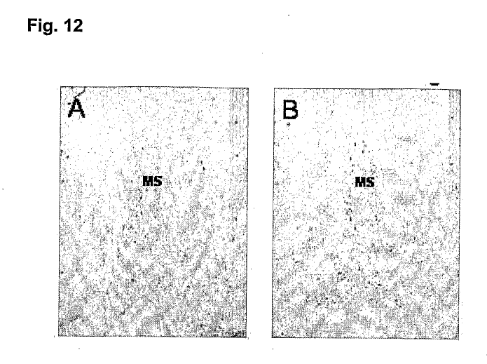

The hystological aspect of the basal forebrain of the VK-AD11 mice revealed the presence of a progressive reduction in neurons that express the cholin-acetyltransferase enzyme (FIG. 11, FIG. 12), measured by means of the anti-cholin-acetyltransferase antiserum (Chemicon).

(6) Hyperiphosphorylation of the Tau Protein and Presence of Intracellular Accumulation

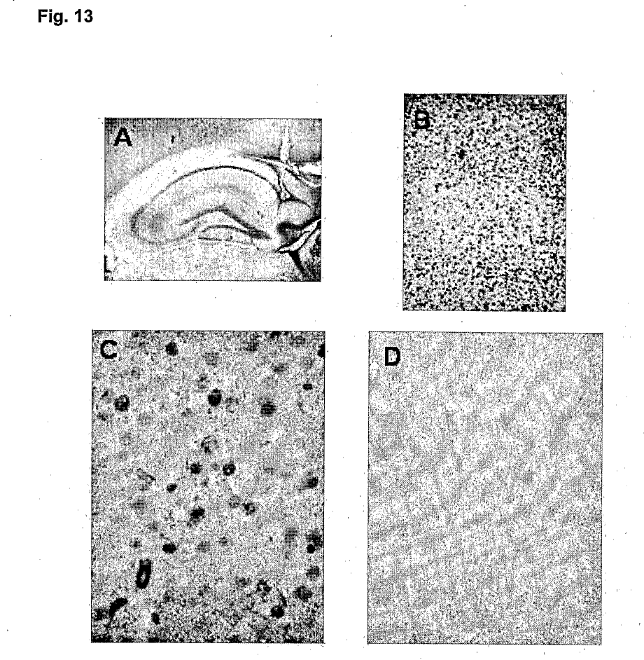

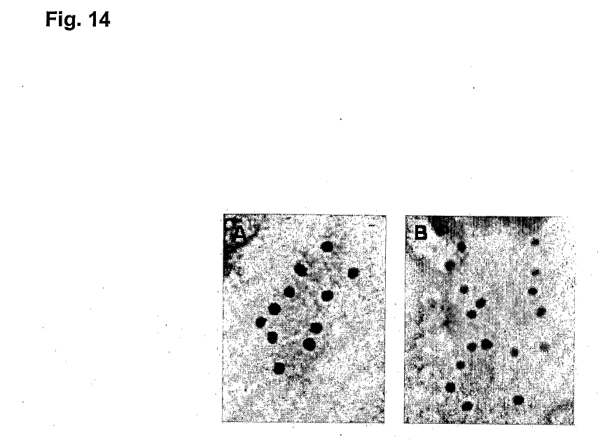

An increase in the expression of the phosphorylated tau protein determined using an antibody (mAb AT8, Innogenetics) directed against the Ser 202 and Ser 205 phosphorylated epitopes of tau (Greenberg and Davies, 1990) is observed. In particular, the protein is expressed in the soma of the neurons of the hippocampus (FIG. 13 A) and of the cortex (FIG. 13B,C), with a perinuclear distribution that is typical of tangles (FIG. 13C). Moreover, the presence of numerous dystrophic neurites is shown (FIG. 13C). Neither structures are present in control mice of the same age (FIG. 13D). Additional experiments, which use the immunohistochemistry technique applied to electronic microscopy, have revealed the presence of protofibrils of tau protein similar to those that constitute filaments that form tangles in Alzheimer's patients (FIG. 14).

(7) Deposition of Extracellular β Amyloid

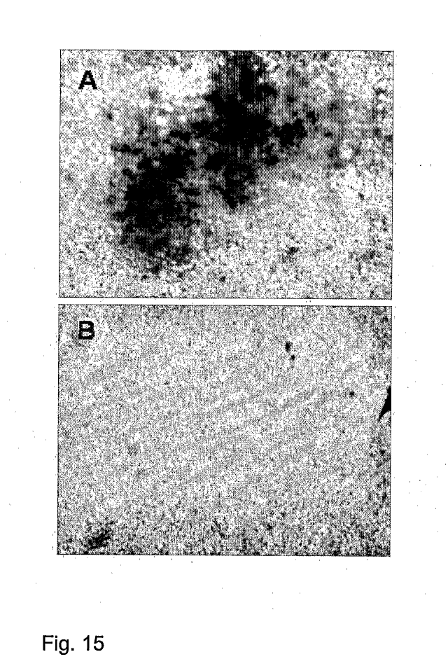

The presence of extracellular aggregates of β-amyloid protein (Aβ) was revealed using the antibody against the Aβ17-24 peptide (mAb 4G8, Signet), the Aβ1-40 peptide (Sigma) and the Aβ1-42 peptide (Biosource). The experiments were conducted using immunohistochemistry techniques. The results have revealed that, at 15 months of age, β-amyloid plaques are present in the cortex and in the hippocampus of VK-AD11 mice (FIG. 15). These plaques occupy a significant part of the surface of the hippocampus with 13% of the surface with respect to a value of 0.1% in control mice of the same age.

(8) Behavioral Deficit

Behavioral analysis was performed on mice of between 2 and 8 months of age (n=6 per experimental group). 2 tests were performed: (i) spatial orientation; (ii) object discrimination.

(i) Spatial Orientation (Test of the Radial Labyrinth with 8 Arms)

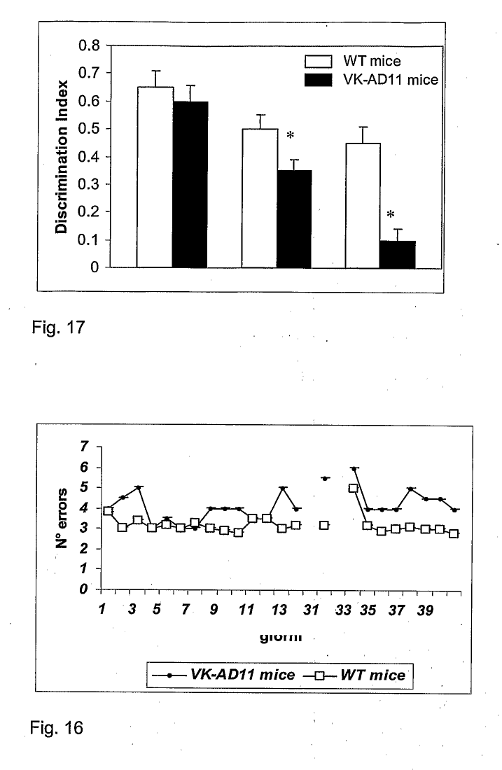

a. learning phase: this consists of filling the same 4 arms with food for 14 days and allowing the mice to familiarize themselves with the labyrinth and learn the position of the food in the different arms of the labyrinth. The test is repeated twice a day and terminated when the mice have found all the food or when 25 entrances in the arms of the labyrinth were found. At 4 months of age, VK-AD11 mice make more mistakes during the initial learning phase (two-way RMANOVA test, p<0.05), but the final level of learning does not differ from that of the control mice. At 8 months of age, the test differs significantly also in the final part of the learning curve (FIG. 16).

b. retention phase: this consists in suspending the test for 31 days and then in resuming it. Control mice retain the notions acquired during the learning phase, while VK-AD11 mice, both at 4 and at 8 months of age, are not able to remember what they learned previously. The learning curves between control mice and VK-AD11 mice were compared by means of two-way ANOVA test (FIG. 16).

c. phase of transferring the notions learned to a new situation: in this case, different arms from those used during the learning phase are filled with food. At both ages, VK-AD11 mice exhibit a behavioral deficit with respect to controls of the same age, which lasts even 5 days after the begining of the learning phase (p<0.01, two way RMANOVA test) (FIG. 16).

(ii) Object discrimination test. The test consists in allowing mice to explore two white cubes, contained in a labyrinth, for 10 min. When the mice are removed from the labyrinth, and one of the cubes is coated with white and black checkered paper. After 1 hour from the end of the first trial, the mice were placed back into the labyrinth to explore the two cubes for 10 additional minutes. The VK-AD11 mice show a reduction in short term memory, not being able to distinguish differently colored cubes (FIG. 17).

In conclusion, VK-AD11 transgenic mice that express the anti-NGF neutralizing antibody recapitulate at the level of the Central Nervous System and of the peripheral innervation many of the typical alterations of neurodegenerative diseases, and in particular of Alzheimer's disease.

### EXAMPLE 3

### Reversal of the Cholinergic Phenotype of Tau Hyperphosphorylation and of β-Amyloid Accumulation by NGF Administration

All experiments were conducted in mice starting from 4 months of age, when neurodegeneration is not so readily apparent. NGF was administered by intranasal injection (Frey et al., 1997) conducted every 2 days. NGF was administered as a 10 μM solution in phosphate buffer pH 7.4, injecting 3 μl per nostril every 2 min and alternating nostrils. The VK-AD11 control mice and non transgenic mice were treated only with phosphate buffer. For each administration, the infusion lasted 30 min. This non invasive method for administering NGF allows to avoid the use of the intraventricular injections to apply NGF directly to the cerebral tissue.

To verify the administration of NGF, the mice were sacrificed 2 months from the begining of the treatment. The brain was removed and fixed in paraformaldehyde to conduct histological analyses.

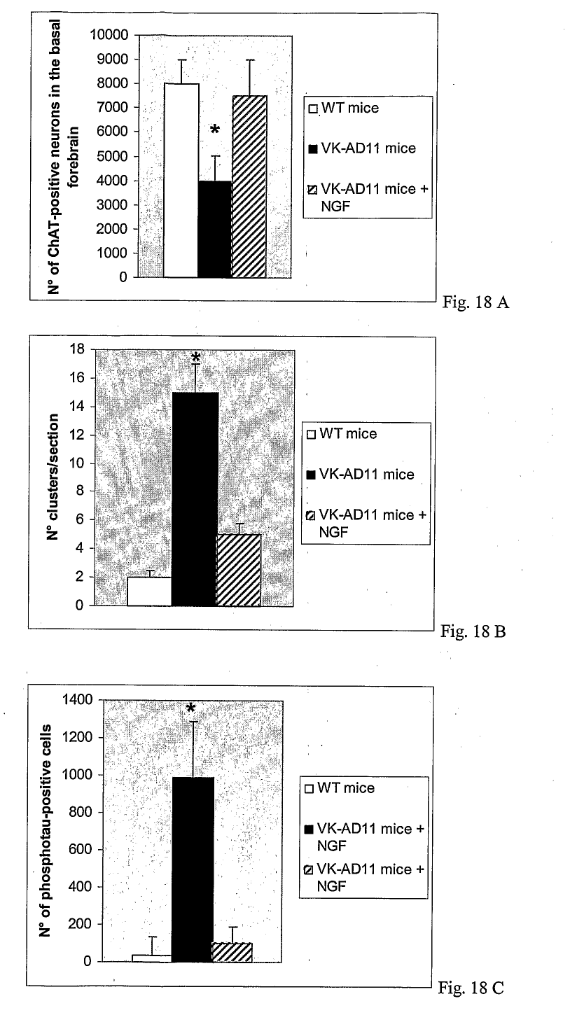

It was possible to observe that, in all injected animals, a similar phenotype to that of the non transgenic control mice was re-established, both with regard to the cholinergic deficit (FIG. 18A), and the deposition of β-amyloid (FIG. 18B) and of hyperphosphorylated tau (FIG. 18C).

### EXAMPLE 4

### Reproductive Ability of the VK-AD11 Mice

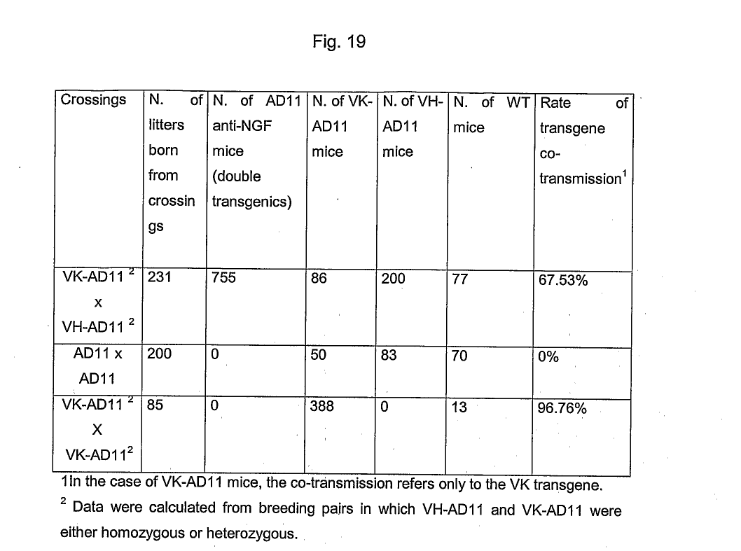

To evaluate the possibility that VK-AD11 mice, unlike AD11 mice, are able to yield as progeny new lines of mice which express not only an anti-NGF antibody, but which are transgenic also for other genes of interest for Alzheimer's disease or of other pathologies, it was decided to analyze the reproductive ability of both mice lines. FIG. 19 shows how VK-AD11 mice, with respect to anti-NGF AD11 mice (derived from the crossing between VH-AD11 and VK-AD11) are surprisingly able to reproduce far more easily and allow to have a homozygous line of VK-AD11 animals. This greater reproductive ability of VK-AD11 mice is important for 2 reasons (1). It is easy to obtain a line of mice with Alzheimer phenotype without having to re-cross the same mice with VH-AD11 mice (2). These VK-AD11 mice can be used for additional crossings with other knock-out mice, thereby obtaining new transgenic mice lines.

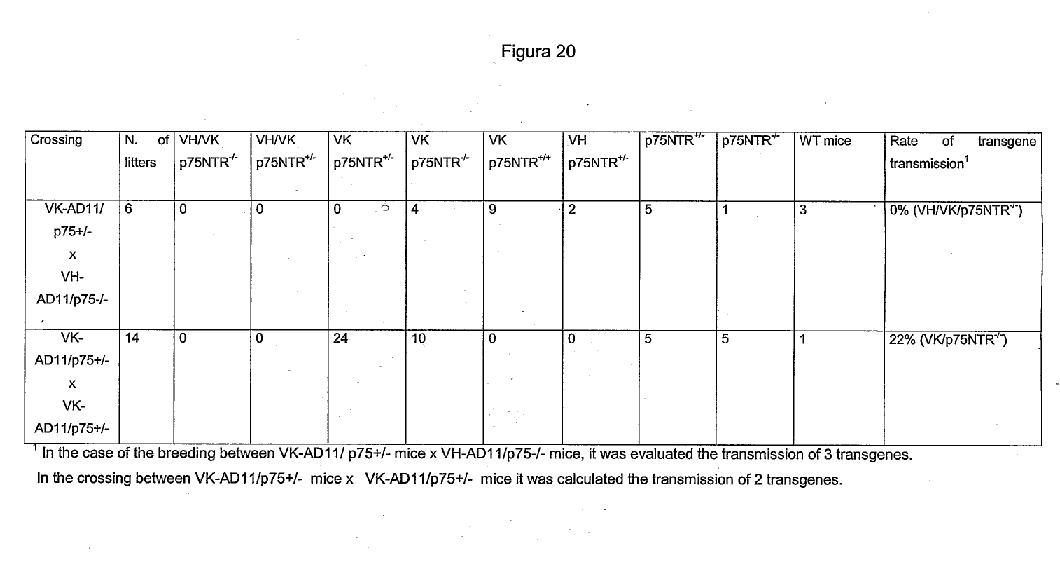

In order to further validate the use of the VK-AD11 mice to produce new lines of transgenic mice, VH-AD11 mice and VK-AD11 mice were crossed with homozygous mice knockout for the p75NTR NGF receptor gene (mice p75NTR−/−; Lee et al., 1992). This receptor is involved in Alzheimer's disease since its reduced expression was observed in the basal forebrain of patients affected by Alzheimer's disease (Mufson et al., 2002) and since, in many cellular lines, it is an apoptosis mediator (Gentry et al., 2004). It was therefore of interest to obtain transgenic mice in which the neurodegenerative effect induced by the anti-NGF antibodies were studied in the genetic context of a knock-out mice for the p75NTR receptor. To obtain the mice that express an anti-NGF antibody and that are simultaneously homozygous knockouts for p75NTR, two different approaches were followed in parallel: (1) in the first case, mice p75NTR^{-/-} (Jackson Laboratories) were crossed respectively with VH-AD11 and VK-AD11 mice to obtain, respectively, the VH-AD11-p75NTR^{-/-} line and the VK-AD11-p75NTR^{-/-} line. These new lines were then crossed between themselves, in order to obtain AD11 anti-NGF/p75NTR^{-/-} mice. This crossing failed to yield positive results, because it was impossible to obtain mice that express both chains of the anti-NGF antibody and that are simultaneously knockouts for p75NTR (FIG. 20), because of the poor reproductive ability of the anti-NGF AD11 mice. (2) In the second approach, the crossing between VK-AD11 and p75NTR^{-/-} easily allows to obtain VK-AD11/p75NTR^{-/-} mice (FIG. 20) that allow to study the consequences of the knocking out of the receptor p75ko on the Alzheimer's phenotype shown by the VK-AD11 mice. This demonstrates that the VK-AD11 mice can easily be crossed with any other transgenic or knock-out mouse, thereby generating new experimental models.

### EXAMPLE 5

### Method for Diagnosing Alzheimer's Disease

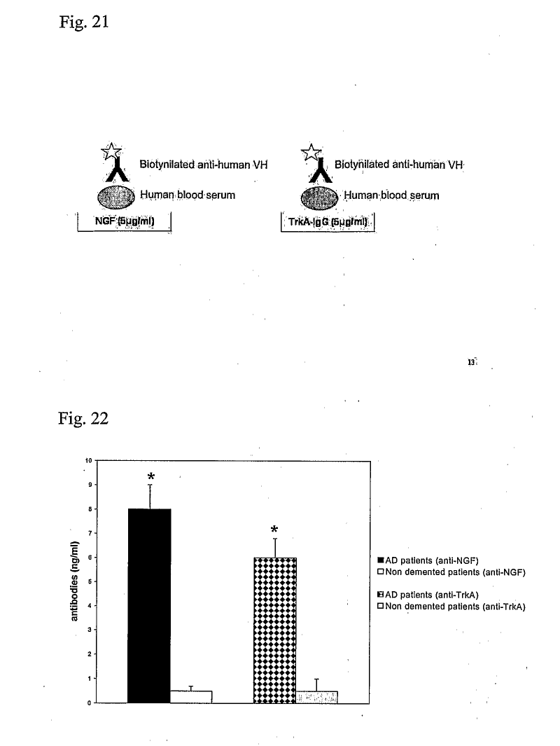

The diagnosis method consists of a system based on the detection of antibodies directed against NGF protein or its TrKA receptor. The method is outlined in FIG. 21. To perform this analysis, human recombinant NGF (Alomone labs, 5 μg/ml) or the immunoadhesin TrkA-IgG (prepared in accordance with Pesavento et al., 2000, 5 μg/ml) were incubated in a 96 well ELISA plate (Nunc Maxisorp). After washings in PBS+0.05% Tween 20, the plates were incubated with sera of 5 patients affected by Alzheimer's disease and of 6 patients of the same age, not affected with any form of dementia. To detect the presence of anti-NGF or anti-TrkA antibodies, the plates were incubated with biotinylated antibodies directed against the heavy chain of human IgG. This method has allowed to detect the presence of antibodies with a variable concentration between 0.2 and 50 ng/ml (FIG. 22).

### BIBLIOGRAPHY

1. Abbas A K, Lichtman A H, Pober J S. 2000. Cellular and Molecular Immunology. Saunders Company, Philadelphia.
2. Allen N D. 1987. In Mammalian development: A practical approach (M. Monk ed.) IRL Press, Washington D.C., 217-234.
3. Angeletti P U, Levi-Montalcini R. 1971. Rev Eur Etud Clin Biol 16:866-874.
4. Bartus R T, Emerich D F. 1999. Jama 282:2208-2209.
5. Bartus R T, et al. 1982. Science 217:408-414.
6. Borchelt D R, et al. 1997. Neuron. 19: 939-945.
7. Capsoni S, Giannotta S, Cattaneo A. 2002a. Mol Cell Neurosci 21:15-28.
8. Capsoni S, Giannotta S, Cattaneo A. 2002b. Proc Natl Acad Sci U S A 99:12432-12437.
9. Capsoni S, Giannotta S, Cattaneo A. 2002c. Brain Aging 2, 24-43.
10. Capsoni S, et al. 2000a. J Neurosci Res 59:553-560.
11. Capsoni S, et al. 2000b. Proc Natl Acad Sci U S A 97:6826-6831.
12. Cattaneo A, Rapposelli B, Calissano P. 1988. J Neurochem. 50:1003-10.
13. Casaccia-Bonnefil P, Kong H, Chao M V. 1998. Cell Death Differ 5:357-364.
14. Chen K S, et al. 1997. J Neurosci 17:7288-7296.
15. Connor B, Dragunow M. 1998. Brain Res Brain Res Rev 27:1-39.
16. Crowley C, et al. 1994. Cell 76:1001-1011.
17. Davis P K, Johnson G V. 1999. J Biol Chem 274:35686-35692.
18. Domenici L, Cellerino A, Maffei L. 1993. Proc R Soc Lond B Biol Sci 251:25-31.
19. Frey W H, et al. 1997. Drug Delivery 4:87-92.
20. Gasparini L, et al. 1998. FASEB J 12:17-34.
21. Gorin P D, Johnson E M. 1979. Proc Natl Acad Sci U S A 76:5382-5386.
22. Gorin P D, Johnson E M, Jr. 1980. Dev Biol 80:313-323.
23. Gotz J. 2001. Tau and transgenic animal models. Brain Res Brain Res Rev 35:266-286.
24. Gotz J, et al. 2001. Science 293:1491-1495.
25. Greenberg S G, Davies P. 1990. Proc Natl Acad Sci USA 87: 5827-5831.
26. Hefti F. 1986. J Neurosci 6:2155-2162.
27. Janus C, et al. 2001. Curr Neurol Neurosci Rep 1:451-457.
28. Kalaria R N. 1999. Ann N Y Acad Sci 893:113-125.
29. Lee K F, et al. 1992. Cell 69:737-749.
30. Levi-Montalcini R. 1952. Ann N Y Acad Sci 55: 330-343.
31. McGeer P L, McGeer E G. 2001. I Neurobiol Aging 22:799-809.
32. Mobley W C, et al. 1986. Brain Res 387:53-62.
33. Molnar M, et al. 1998. Eur J Neurosci 10:3127-3140.
34. Oddo S, et al. 2003. Neuron 39: 409-421.
35. Pesavento E, et al. 2002. Eur J Neurosci 15:1030-1036.
36. Pesavento E, et al. 2000. Neuron 25:165-175.
37. Ruberti F, et al. 2000. J Neurosci 20:2589-2601.
38. Scott S A, et al. 1995. J Neurosci 15:6213-6221.
39. Selkoe D J. 2001 Physiol Rev 81:741-766.
40. Mufson E J, et al. 2002. J Comp Neurol. 443:136-153.
41. Gentry J J, Barker P A, Carter B D. 2004. Prog Brain Res. 146:25-39.

1-12. (canceled)

13. Non human transgenic animal able to express ubiquitarily an anti-NGF neutralizing antibody wherein said antibody is composed by an endogenous VH chain and by an exogenous VK chain.

14. Non human transgenic animal according to claim 13, wherein the exogenous VK chain is that of the AD11 antibody, having essentially the amino acid sequence of Seq Id No. 1.

15. Non human transgenic animal according to claim 13 belonging to the murine genus.

16. Non human transgenic animal according to claim 15 belonging to the Mus musculus species.

17. A method of identifying compounds with therapeutic activity, which comprises using the non human transgenic animal according of claim 13 as an animal model to identify compounds with therapeutic activity.

18. The method of claim 17, wherein the therapeutic activity comprises neurodegenerative pathologies.

19. A method of obtaining a line of transgenic animals with at least two transgenes, which comprises: crossing the non human transgenic animal according to claim 13 with a second non human transgenic animal, and obtaining a line of non human transgenic animals with at least two transgenes, wherein the first transgene codifies for the exogenous VK chain of an anti-NGF antibody and the second one for a different transgene, wherein said transgenes codify for functions involved in a pathology.

20. The method of claim 19, wherein said transgenes codify for functions involved in neurodegenerative pathologies.

21. The method of claim 20, wherein the second non human transgenic is an homozygous “knockout” for the p75NTR, NGF receptor gene, or for a part thereof.

22. A method for the early prognosis and/or diagnosis of neurodegenerative disease, comprising: drawing of a peripheral biological fluid from a patient and detecting in said fluid an antibody selected from the group consisting of anti-NGF, anti-TrkA, and antibodies against proteins linked to NGF activity.

23. The method according to claim 22 wherein the peripheral biological fluid is blood, serum or urine.

24. The method according to claim 22, wherein the neurodegenerative disease is Alzheimer's disease.
